(19) United States
(12) Patent Application Publication
(10) Pub. No.: US 2025/0249250 A1
(43) Pub. Date: Aug. 7, 2025

(54) SYSTEM AND METHOD FOR VAGUS NERVE STIMULATION

(71) Applicant: THE ALFRED E. MANN FOUNDATION FOR SCIENTIFIC RESEARCH, VALENCIA, CA (US)

(72) Inventor: Jason GOLDBERG, Oakland, CA (US)

(21) Appl. No.: 19/043,158

(22) Filed: Jan. 31, 2025

Related U.S. Application Data
(60) Provisional application No. 63/548,729, filed on Feb. 1, 2024.

(51) Int. Cl. A61N 1/36

(52) U.S. Cl. CPC A61N 1/36053; A61N 1/36139

(57) ABSTRACT

A system and method for vagus nerve stimulation. In some embodiments, a method includes: receiving a motion signal from a motion sensor of an implantable device implanted in a subject; generating, from the motion signal, a calculated biomarker; detecting an epileptic seizure, the detecting being based on the calculated biomarker; and in response to the detecting of the epileptic seizure, applying, by the implantable device, vagus nerve stimulation.

### CROSS-REFERENCE TO RELATED APPLICATION(S)

The present application claims priority to and the benefit of U.S. Provisional Application No. 63/548,729, filed Feb. 1, 2024, entitled “SYSTEMS AND METHODS FOR SEIZURE DETECTION”, the entire content of which is incorporated herein by reference.

### FIELD

One or more aspects of embodiments according to the present disclosure relate to vagus nerve stimulation, and more particularly to closed-loop vagus nerve stimulation.

### BACKGROUND

Epileptic seizures may occur in subjects with epilepsy with little warning, and may result in undesirable sequelae.

It is with respect to this general technical environment that aspects of the present disclosure are related.

### SUMMARY

According to an embodiment of the present disclosure, there is provided a method, including: receiving a motion signal from a motion sensor of an implantable device implanted in a subject; generating, from the motion signal, a calculated biomarker; detecting an epileptic seizure, the detecting being based on the calculated biomarker; and in response to the detecting of the epileptic seizure, applying, by the implantable device, vagus nerve stimulation.

In some embodiments: the calculated biomarker includes a calculated heart rate or a calculated respiration rate; and the detecting, based on the calculated biomarker, of the epileptic seizure, includes detecting the epileptic seizure based on an increase or decrease in the calculated heart rate or based on an increase or decrease in the calculated respiration rate.

In some embodiments: the calculated biomarker includes a calculated respiration rate; and the detecting, based on the calculated biomarker, of the epileptic seizure, includes detecting the epileptic seizure based on an increase or decrease in the calculated respiration rate.

In some embodiments: the detecting of the epileptic seizure includes detecting a decrease in the calculated respiration rate, and the detecting of the decrease in the calculated respiration rate includes performing frequency tracking of the calculated respiration rate with an infinite impulse response adaptive notch filter tuned to the calculated respiration rate.

In some embodiments: the generating of the calculated biomarker includes calculating a heart rate; and the detecting, based on the calculated biomarker, of the epileptic seizure, includes detecting the epileptic seizure based on an increase in the calculated heart rate.

In some embodiments, the calculating of the heart rate includes performing a method selected from the group consisting of linear filtering, numerical differentiation, application of a memoryless nonlinear transform, low pass filtering, peak detection, and combinations thereof.

In some embodiments, the method further includes: detecting an increase in a heart rate of the subject; determining that the subject is engaged in exercise; and determining, based on the increase in the heart rate, and based on the determining that the subject is engaged in exercise, that an epileptic seizure is not occurring.

In some embodiments, the method further includes detecting, based on the motion sensor, muscle movements characteristic of an epileptic seizure, wherein the detecting of the epileptic seizure is further based on the detecting of the muscle movements.

In some embodiments, the detecting of the muscle movements includes detecting a period of high amplitude signals in a frequency band characteristic of shaking encountered during clonic seizures.

In some embodiments, the method further includes receiving a magnetic field signal from a magnetometer of the implantable device, wherein the detecting of the epileptic seizure is further based on the detecting of the magnetic field signal.

In some embodiments, the method further includes detecting, based on the motion sensor, motion characteristic of poor sleep quality, wherein the detecting of the epileptic seizure is further based on the detecting of the motion characteristic of poor sleep quality.

In some embodiments, the detecting of the motion characteristic of poor sleep quality includes detecting motion corresponding to a position change of the subject while the subject is lying down.

In some embodiments: the detecting of the epileptic seizure includes detecting the calculated biomarker passing a threshold, and the threshold is based on a history of the calculated biomarker.

In some embodiments, the detecting of the epileptic seizure includes detecting of the epileptic seizure by a machine learning model, based on a plurality of signals including the motion signal.

In some embodiments, the plurality of signals further includes a magnetic field signal.

In some embodiments, the method further includes training the machine learning model by performing supervised training with training data including a plurality of labeled data elements, each labeled data element being labeled with an indicator of whether a seizure was occurring when the data element was collected.

In some embodiments, the implantable device includes a housing having a biocompatible outer surface and containing the motion sensor and a vagus nerve stimulation circuit.

In some embodiments, the motion sensor is a micro-electromechanical systems (MEMS) sensor.

In some embodiments, the motion sensor includes an accelerometer.

In some embodiments, the motion sensor includes a gyroscope.

According to an embodiment of the present disclosure, there is provided a system, including: an implantable device, including: a motion sensor; a vagus nerve stimulation circuit; and a processing circuit, the processing circuit being configured to: receive a motion signal from the motion sensor, generate, from the motion signal, a calculated biomarker; detect an epileptic seizure, the detecting being based on the calculated biomarker; and in response to the detecting of the epileptic seizure, apply vagus nerve stimulation.

In some embodiments: the calculated biomarker includes a calculated heart rate or a calculated respiration rate; and the detecting, based on the calculated biomarker, of the epileptic seizure, includes detecting the epileptic seizure based on an increase or decrease in the calculated heart rate or based on an increase or decrease in the calculated respiration rate.

In some embodiments: the calculated biomarker includes a calculated respiration rate; and the detecting, based on the calculated biomarker, of the epileptic seizure, includes detecting the epileptic seizure based on an increase or decrease in the calculated respiration rate.

In some embodiments: the detecting of the epileptic seizure includes detecting a decrease in the calculated respiration rate, and the detecting of the decrease in the calculated respiration rate includes performing frequency tracking of the calculated respiration rate with an infinite impulse response adaptive notch filter tuned to the calculated respiration rate.

In some embodiments: the generating of the calculated biomarker includes calculating a heart rate; and the detecting, based on the calculated biomarker, of the epileptic seizure, includes detecting the epileptic seizure based on an increase in the calculated heart rate.

In some embodiments, the calculating of the heart rate includes performing a method selected from the group consisting of linear filtering, numerical differentiation, application of a memoryless nonlinear transform, low pass filtering, peak detection, and combinations thereof.

In some embodiments, the implantable device is configured to be implanted in a subject, and the processing circuit is further configured to: detect an increase in a heart rate of the subject; determine that the subject is engaged in exercise; and determine, based on the increase in the heart rate, and based on the determining that the subject is engaged in exercise, that an epileptic seizure is not occurring.

In some embodiments, the processing circuit is further configured to detect, based on the motion sensor, muscle movements characteristic of an epileptic seizure, wherein the detecting of the epileptic seizure is further based on the detecting of the muscle movements.

In some embodiments, the detecting of the muscle movements includes detecting a period of high amplitude signals in a frequency band characteristic of shaking encountered during clonic seizures.

In some embodiments: the processing circuit is further configured to receive a magnetic field signal from a magnetometer of the implantable device, and the detecting of the epileptic seizure is further based on the detecting of the magnetic field signal.

In some embodiments: the processing circuit is further configured to detect, based on the motion sensor, motion characteristic of poor sleep quality; and the detecting of the epileptic seizure is further based on the detecting of the motion characteristic of poor sleep quality.

In some embodiments, the implantable device is configured to be implanted in a subject, and the detecting of the motion characteristic of poor sleep quality includes detecting motion corresponding to a position change of the subject while the subject is lying down.

In some embodiments: the detecting of the epileptic seizure includes detecting the calculated biomarker passing a threshold, and the threshold is based on a history of the calculated biomarker.

In some embodiments, the detecting of the epileptic seizure includes detecting of the epileptic seizure by a machine learning model, based on a plurality of signals including the motion signal.

In some embodiments, the plurality of signals further includes a magnetic field signal.

In some embodiments, the processing circuit is further configured to train the machine learning model by performing supervised training with training data including a plurality of labeled data elements, each labeled data element being labeled with an indicator of whether a seizure was occurring when the data element was collected.

In some embodiments, the implantable device includes a housing having a biocompatible outer surface and containing the motion sensor and the vagus nerve stimulation circuit.

In some embodiments, the motion sensor is a micro-electromechanical systems (MEMS) sensor.

In some embodiments, the motion sensor includes an accelerometer.

In some embodiments, the motion sensor includes a gyroscope.

### BRIEF DESCRIPTION OF THE DRAWINGS

These and other features and advantages of the present disclosure will be appreciated and understood with reference to the specification, claims, and appended drawings wherein:

FIG. 1 is a block diagram of an implantable device, according to an embodiment of the present disclosure;

FIG. 2 is a block diagram of a system and method for performing closed-loop vagus nerve stimulation, according to an embodiment of the present disclosure;

FIG. 3A is a portion of a flowchart of a method, according to an embodiment of the present disclosure; and

FIG. 3B is a portion of a flowchart of a method, according to an embodiment of the present disclosure.

### DETAILED DESCRIPTION

The detailed description set forth below in connection with the appended drawings is intended as a description of exemplary embodiments of a system and method for vagus nerve stimulation provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

Epilepsy is a group of neurological disorders characterized by recurrent seizures. A patient, or “subject” with epilepsy may experience seizures at unpredictable times, either with no identifiable trigger, or, in some cases, triggered by an external stimulus. During a seizure, the subject may exhibit a sudden increase in heart rate, referred to as ictal tachycardia, or a decrease in respiration rate, which may be referred to as ictal apnea, or abrupt muscle contractions or spasms, or a combination of such symptoms. Poor sleep or the presence of a strong magnetic field may increase the likelihood of a seizure.

Seizures (e.g., clonic seizures) may be mitigated using vagus nerve stimulation. Vagus nerve stimulation may involve applying an electrical stimulus to the vagus nerve; this stimulus may propagate to the brain and lessen the severity of a seizure, prevent a seizure, or stop a seizure that is in progress. For example, in an intervention referred to as open loop vagus nerve stimulation, the vagus nerve is periodically stimulated (e.g., for 5 seconds every 30 seconds) in an ongoing manner. In an intervention referred to as on-demand vagus nerve stimulation, the vagus nerve is stimulated in response to user input, e.g., a button press or a screen click performed by the subject or by an assistant or a clinician. In an intervention referred to as closed-loop vagus nerve stimulation, the vagus nerve is stimulated automatically (e.g., without user intervention) in response to the detection, by a control system, that a seizure has started or is incipient.

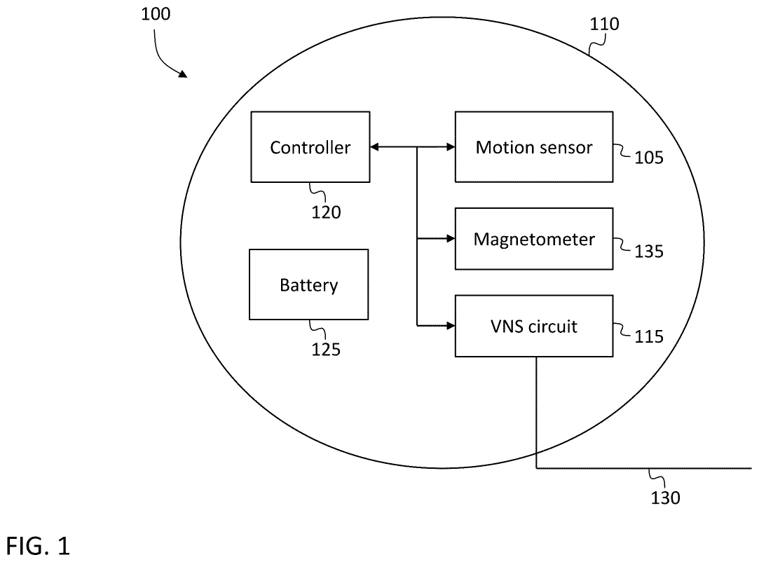

Closed-loop vagus nerve stimulation may be performed by an implantable device, such as the implantable device 100 illustrated in FIG. 1, which may be implanted in the chest or neck of the subject. In some embodiments, a motion sensor 105 in the implantable device 100 may be employed for the detection of seizures. The motion sensor 105 may include, for example, one or more accelerometers (e.g., three accelerometers, measuring acceleration along three different axes (e.g., along three orthogonal axes), or one or more gyroscopes (e.g., three gyroscopes, measuring rotation rate along three different axes (e.g., along three orthogonal axes). As used herein, a “motion sensor” is a device that senses motion. As such, a single accelerometer is a motion sensor, and a set of three orthogonal accelerometers is also a motion sensor (and, as illustrated by this example, a motion sensor may include one or more motion sensors). The motion sensor 105 may be packaged in a housing 110 (discussed in further detail below) along with other components of the implantable device 100.

The motion sensor may produce signals from which various biomarkers may be calculated. For example, the motion sensor may be used to generate so-called seismocardiograms or gyrocardiograms from which instantaneous heart rate (HR) may be calculated. The same sensor signals may be used to calculate the instantaneous respiration rate (RR) as well. One exemplary method for extracting the respiration rate is to low-pass filter the inertial sensor signals, since motion due to breathing happens on a longer time scale than mechanical vibrations due to the heart. A neck mounted or implanted motion sensor may be used to measure pulsing of blood through the carotid artery. As used herein, a “biomarker” is an indicator of one or more aspects of the biological state or condition of the subject. As such, a biomarker may be the subject's heart rate, or a biomarker may be the subject's heart rate and respiration rate.

Each of the accelerometers and gyroscopes may be a microelectromechanical systems (MEMS) sensor, e.g., fabricated on a semiconductor chip using photolithographic or analogous methods, and including, e.g., a cantilevered beam that may deflect when the motion sensor 105 is subject to acceleration, or a resonant structure within which energy may couple between different resonant modes when the motion sensor 105 is rotated.

In some embodiments, the motion sensor 105 is packaged with (e.g., in the same housing as) a circuit for performing vagus nerve stimulation (or VNS circuit) 115. The housing 110 may be implantable (e.g., it may be hermetically sealed and its outer surface may be composed of a biocompatible material). The implantable device 100 may include (i) the motion sensor 105 (ii) the vagus nerve stimulation circuit 115, (iii) a controller 120, which may be or include a processing circuit (discussed in further detail below) for controlling the other circuits of the implantable device 100, (iv) a battery 125 for powering the circuits of the implantable device 100 and (v) the housing 110. One or more electrode wires 130 may extend from the implantable device 100 and near (e.g., the electrode wires 130 may have an end wrapped around) the vagus nerve of the subject, for providing vagus nerve stimulation. The battery 125 may be rechargeable (e.g., using power inductively coupled to the implantable device 100 through the skin of the user) or it may be a single-use battery that is replaced as needed (e.g., when the entire implantable device 100 is replaced).

In some embodiments, a magnetometer 135 is included in the implantable device 100. The magnetometer 135 may be or include a MEMS sensor, e.g., including a flexible current-carrying element that deforms as a result of the Lorentz force exerted on the element when current flows through it in a magnetic field. In other embodiments the magnetometer 135 may be or include a different kind of sensor, e.g., a Hall effect sensor a magneto-diode, or a magneto-transistor. In some embodiments, the magnetometer 135 includes a plurality of magnetometers, e.g., the magnetometer 135 may be a three-axis magnetometer including three magnetometers sensing the magnetic field along three substantially orthogonal axes. As used herein, a sensor including a plurality of sensors (e.g., a three-axis accelerometer including three accelerometers or a three-axis magnetometer including three magnetometers) may be referred to as a “compound” sensor, and a sensor consisting of a single sensor (e.g., a single-axis accelerometer) may be referred to as a “simple” sensor.

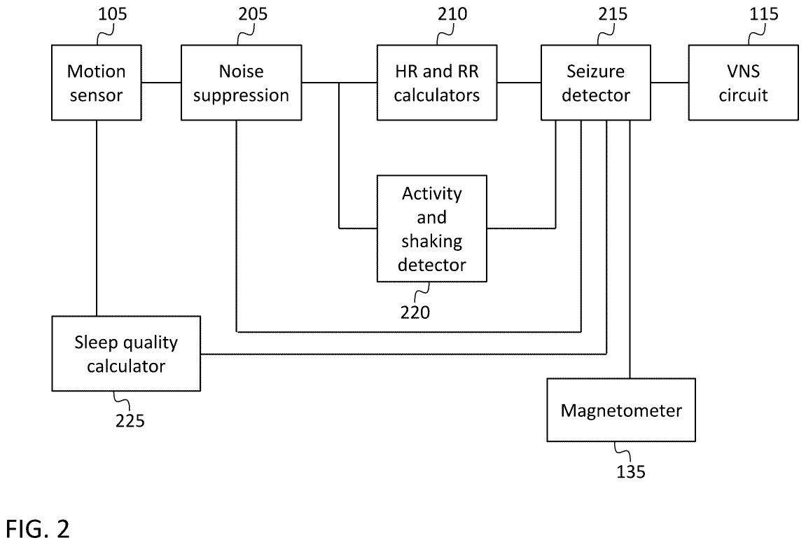

The signals produced by the motion sensor 105 may be processed to perform seizure detection and closed-loop vagus nerve stimulation as illustrated in FIG. 2. The signal from the motion sensor 105 may be processed by a noise suppression circuit, or “denoiser” 205 and fed to heart rate and respiration rate calculators 210 (which may include a heart rate calculator, a respiration rate calculator, or both). The calculated heart rate or respiration rate may then be fed to a seizure detector 215, which may determine whether a seizure is occurring. If the seizure detector 215 determines that a seizure is occurring, it may cause the vagus nerve stimulation circuit 115 to perform (closed-loop) vagus nerve stimulation.

The noise suppression circuit 205 may perform several functions, including (i) detecting the presence of severe motion artifacts (which may be referred to as “motion artifact detection”) and (ii) attenuating smaller artifacts and noise. Motion artifact detection may involve detecting motion artifacts by computing a noisy measure of instantaneous signal magnitude (which may be based on multiple sensing axes), smoothing the resulting sequence of magnitudes with a low pass filter, and comparing the smoothed magnitude to a threshold. If the smoothed magnitude exceeds the threshold, the noise suppression circuit 205 may declare motion artifacts (e.g., notify the heart rate and respiration rate calculators 210, the activity and shaking detector 220, or the seizure detector 215 of the presence of motion artifacts); the declaration of motion artifacts may remain in effect for some minimum period of time. Such a notification may prevent the seizure detector 215 from detecting a seizure.

The attenuating of smaller artifacts and noise may be performed by linear filtering that suppresses the received signal over frequency bands known not to contain desired signal components. Such a filter may take the form of a simple high pass filter since motion artifacts often occupy a frequency band beneath that of a seismocardiogram. Respiration signals may occupy a much lower frequency range, and a low pass filter may be employed for noise suppression when the respiration rate is being calculated. In some embodiments, the noise suppression circuit 205 may perform band-pass filtering, or it may include non-linear filtering elements such as an artifact detection and suppression circuit or algorithm, which may, for example, detect and eliminate pulses larger than a threshold.

Heart rate may be calculated from a seismocardiogram signal by computing successive differences of detection times associated with some fiducial point in the seismocardiogram (such as the time of aortic opening). These detection times may be obtained using a method analogous to the Pan-Tompkins method that may be used for electrocardiograms. Several variants are possible, including: (i) the use of a linear filter to suppress energy outside of a band known to contain the desired signal, (ii) (numerical) differentiation of the resulting signal (e.g., successive differencing of adjacent samples), (iii) the application of a memoryless, nonlinear transform, such as a squaring operation, to enhance peak magnitudes, (iv) the use of low pass filtering to produce a smoothed peak, or (v) peak detection by comparison to a threshold while requiring successive detections to be located in time within physiologically reasonable limits (e.g., disregarding a second peak that is detected less than 300 ms after a first peak, or inserting a peak, on the assumption that a peak has been missed, if a second peak is found more than 1.4 seconds after a first peak).

As such, heart rate calculation may be performed based on the signal from the motion sensor by, e.g., filtering the motion sensor signal with a filter, or a series of filters, that emphasizes or amplifies signal components associated with motion due to cardiac activity. In some embodiments, a filter that is analogous to a Pan-Tompkins algorithm is used. The filter may include low-pass filtering (to the extent such filtering is not already performed by the noise suppression circuit 205), high-pass filtering (to suppress low frequency noise or low frequency motion (e.g., caused by the subject walking or changing positions)) or non-linear filtering such as squaring of the signal. Frequency domain analysis may be used to calculate the heart rate (e.g., by taking a Fourier transform of the motion sensor signal and finding the largest peak (within a range of frequencies corresponding to plausible heart rates) in the resulting frequency-domain signal. Respiration rate calculation may be performed in an analogous manner, e.g., by taking a Fourier transform of the motion sensor signal and finding the largest peak (within a range of frequencies corresponding to plausible respiration rates) in the resulting frequency-domain signal.

The calculation of respiration rate may be performed using a method based on the observation that the quasi-periodic low-frequency respiration signal is dominated by its fundamental frequency. As such, the respiration rate calculation problem may be posed as one of frequency tracking. One frequency tracking method which may be used is that of an infinite impulse response (IIR) adaptive notch filter tuned to notch out the time-varying fundamental frequency of the respiration signal.

The seizure detector may calculate the likelihood that a seizure is occurring, and make the determination that a seizure is occurring when this likelihood exceeds a threshold. The likelihood calculation may be based on a combination of one or more of the heart rate, the respiration rate, signals from an activity and shaking detector 220, signals from a sleep quality calculator 225, and signals from the magnetometer 135. An increase in heart rate or a decrease in respiration rate may indicate that a seizure is occurring.

The activity and shaking detector 220 may include (linear or nonlinear) filters for detecting (i) motions that are characteristic of exercising and (ii) motions (e.g., shaking of the subject's torso in a way consistent with clonic seizure convulsions), that would result from muscle movements characteristic of a seizure. For example, detection of shaking associated with seizures may be achieved by identifying periods of high amplitude signals in a frequency band characteristic of shaking encountered during clonic seizures. Similarly, an activity such as walking may be detected by identifying periods during which signals in a frequency band characteristic of walking are present.

These motions may decrease or increase the likelihood that a seizure is occurring (with, e.g., exercise being an alternate mechanism for an increase in heart rate, so that if an increase in heart rate is detected, the likelihood that it signals the presence of a seizure is lower if it is accompanied by motion characteristic of exercise). Similarly, if the signal from the activity and shaking detector 220 recently indicated exercise, and at a later time indicates that the exercise has ceased, then the ceasing of exercise may be an alternate mechanism for a reduction in respiration rate.

Strong magnetic fields may cause, or contribute to, the occurrence of an epileptic seizure in some subjects; as such, the seizure detector may use a signal from the magnetometer indicating a strong magnetic field (e.g., a field having a magnitude exceeding a threshold, the threshold being between 20 microteslas and 11.700000 teslas) as a factor increasing the likelihood that a seizure is occurring. In a (compound) magnetometer including several (e.g., three) simple single-axis magnetometers, the magnitude of the magnetic field may be calculated as the magnitude of the vector sum of the magnetic field vectors measured by the magnetometer, each such magnetic field vector being a vector having a magnitude equal to the field strength measured by a respective simple magnetometer of the compound magnetometer and a direction along the sensing axis of the simple magnetometer.

Poor sleep quality may increase the likelihood of a seizure. As such, the calculated likelihood that a seizure is occurring calculated by the seizure detector 215 may be based in part on the recent sleep quality of the subject, as calculated by the sleep quality calculator 225. Sleep quality may be quantified by assessing the frequency with which motion artifacts are detected while a subject is lying down. Determining that the subject is lying down may involve detection of orientation, e.g., using the pitch and roll (defined with respect to a dorsal-ventral axis of the subject) measured by (e.g., by integrating the output of) a three-axis gyroscope to classify the orientation of the subject as upright vs lying down (e.g., left-side lying down, right-side lying down, supine, or prone). During periods of time where a lying-down orientation is detected, the sleep quality calculator 225 may gather statistics about the relative frequency and magnitude of motion artifacts corresponding to shifting positions. Such statistics may be used either directly by the seizure detector 215 to adjust a heart rate threshold or a respiration rate threshold, or incorporated as features in a machine learning algorithm for seizure detection.

For example, if the sleep quality calculator 225 indicates that sleep quality has been poor, during, e.g., the previous 24 hours or 48 hours, then the seizure detector 215 may use this information as a factor increasing the likelihood that a seizure is occurring (and it may, for example, use a lower threshold in determining whether a certain rate of increase in the heart rate indicates that a seizure is occurring).

The blocks illustrated in FIG. 2 may be implemented in some combination of hardware, software, and firmware. For example, each of the motion sensor 105 and the magnetometer 135 may include analog sensors and analog-to-digital converters, and the vagus nerve stimulation circuit 115 may include a digital-to-analog converter for converting a digital signal (received by, or generated by, the vagus nerve stimulation circuit 115) to a suitable analog stimulation signal for the vagus nerve. The remaining blocks of FIG. 2 may be entirely digital, discrete time blocks implemented using one or more processing circuits such as, e.g., finite state machines or stored-program computers.

Various methods or algorithms may be employed by the seizure detector 215 to calculate the likelihood that a seizure is occurring and to make a determination of whether a seizure is occurring (and whether to perform closed-loop vagus nerve stimulation). Detection of seizures may be achieved through a series of statistical tests which attempt to identify changes in heart rate or respiration rate often associated with seizures. A variety of test statistics may be used to quantify how much recent heart rate or respiration rate calculations deviate from short-term recent history. Such test statistics, or “metrics” may include a variety of combinations of minimum absolute or relative changes in rate sufficient to trigger a seizure detection. In particular, the seizure detector 215 may seek to identify large, sudden increases in heart rate (ictal tachycardia) with respect to recent history, or to identify decreases in respiration rate (ictal apnea). Such increases or decreases may be ones that pass (exceed or fall below) a respective threshold based on a recent history of the biomarker (e.g., a threshold that is some factor times an average (e.g., a weighted average) of the N most recently calculated values of the biomarker (N being a positive integer)). As used herein, “passing” a threshold means exceeding the threshold or falling below the threshold. In some embodiments, the thresholds associated with such statistical tests may instead be fixed, or may adapt based on different factors, including the presence of strong magnetic fields. For example, if magnetometer signals with smoothed magnitude exceed some threshold, the threshold for detecting seizures may be relaxed such that seizure is detected more readily under such conditions.

The seizure detector 215 may also accept inputs from the activity and shaking detector 220. If the activity and shaking detector 220 identifies shaking typical of a clonic seizure, then the seizure detector 215 may indicate the presence of a seizure. Alternatively, if the activity and shaking detector 220 identifies significant motion of the type that would likely be associated with exercise, the logic of the seizure detector 215 may be overridden to avoid false detection of seizure due to increased heart rate.

As another example, the seizure detector 215 may be or include a supervised machine learning algorithm (e.g. logistic regression, a support vector machine, or a decision tree) that accepts a feature vector and declares whether or not a seizure is taking place or imminent. Features may include measures of changes in heart rate or respiration rate with respect to recent history, smoothed magnetic field calculations, the raw metrics computed in the activity and shaking detector 220, or features of the sleep quality calculator described below. Labeled training data may be used to train the machine learning algorithm.

As another example, if the activity and shaking detector 220 declares the presence of convulsive type shaking (indicating muscle movements characteristic of an epileptic seizure), the seizure detector 215 may automatically declare the presence of a seizure. Otherwise, the heart and respiration rate calculations may be analyzed to determine whether abnormalities associated with seizures (e.g., ictal tachycardia, ictal bradycardia, or ictal apnea) are present. A hypothesis test may be carried out to decide if there is good evidence of the onset of seizure. The hypothesis test may be repeatedly carried out as new data becomes available.

As another example, each of the inputs to the seizure detector 215 may be (e.g., as a result of processing performed by a respective one of the blocks shown in FIG. 2, or by a respective processing block not shown in FIG. 2) in the form of a signal a higher value in which indicates a greater likelihood that a seizure is occurring. For example, the input corresponding to calculated heart rate may be signal that is proportional to a recent rate of increase of the calculated heart rate. The seizure detector 215 may, in such an embodiment, compare each of the signals it receives to a respective threshold to form a set of bits, and then perform a logical operation on the bits to generate single bit indicating whether a seizure is occurring. As mentioned above, in some embodiments, this threshold is adjusted based on other factors that are correlated with propensity for a seizure to occur, e.g., the threshold may be reduced after a history of poor sleep quality or in the presence of a strong magnetic field. For example, the heart rate calculator may produce a signal representing the rate of increase of the heart rate (e.g., over some number of (e.g., between 3 and 30) cardiac cycles); and the threshold applied by the seizure detector 215 may be some rate (e.g., between 0.1 beats per minute (bpm) per second and 50 bpm per second) of increase of heart rate, which, when exceeded, suggests that a seizure is occurring. Similarly, the respiration rate calculator may produce a signal representing the rate of decrease of the respiration rate (e.g., over some number of (e.g., between 2 and 20) respiration cycles); and the threshold applied by the seizure detector 215 may be some rate (e.g., between 0.1 breaths per minute per second and 50 breaths per minute per second) of decrease of respiration rate, which, when exceeded, suggests that a seizure is occurring. The logical operation used by the seizure detector 215 may be, for example, simple voting (with the single resulting bit being one if a certain number of the input bits have a value of one) or it may use a weighted voting operation, with, e.g., the rate of increase of heart rate being weighted by a weight that is greater (e.g., by a factor between 1.1 and 10.0) than the weight given to any of the other bits.

In some embodiments, the signals received by the seizure detector 215 are combined by a function (e.g., a continuous function of some or all of the input signals) and the output of the function is compared to a threshold to determine whether a seizure is occurring. The function may be, for example, a weighted average of the input signals, or a polynomial of the input signals (where the coefficients of the polynomial may be empirically determined by fitting a polynomial to experimental data of circumstances under which various subjects have experienced seizures.

As another example, each of several primary inputs that may directly suggest that a seizure is occurring (e.g., a measure of increased heart rate, a measure of decreased respiration rate, and a measure of muscle movements characteristic of an epileptic seizure) may be combined (e.g., in a weighted sum) and compared to a threshold, which may be set based on secondary inputs. For example, the threshold may be set lower based on inputs, such as a signal indicating a strong magnetic field, or a signal indicating recent poor sleep quality, that indicate an increased propensity for a seizure to occur, or the threshold may be set higher based on an input indicating that the subject is exercising.

As mentioned above, in some embodiments, the seizure detector 215 includes a machine learning model that receives the input signals and generates a determination of whether a seizure is occurring, or a control signal for the vagus nerve stimulation circuit 115. This machine learning model may be trained using supervised training with a labeled training data set that includes sets of input signals each labeled with (i) an indicator of whether a seizure was occurring when the training data were captured or (ii) an indicator of an appropriate vagus nerve stimulation signal to be used in the circumstances under which the training data were captured. Such training may result in the model being capable of (i) recognizing, e.g., certain signatures, in the signals from the sensors, that correspond to seizures, and of (ii) taking into account factors, such as a strong magnetic field of a recent history of poor sleep, that signal an increased likelihood of a seizure without indicating that a seizure is occurring at a particular time. In some embodiments, the training data includes (e.g., consists of) data previously obtained from the subject. The labeling of the training data may be performed retroactively, e.g., based on reporting, by the subject, of having experienced a seizure.

The machine learning model may be or include a classifier, e.g., for classifying the received inputs as corresponding to (i) a seizure being in progress, or (ii) a seizure not being in progress. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof.

In some embodiments, the implantable device 100 may perform additional functions, such as warning the user (e.g., the subject) (i) that a strong magnetic field is present, increasing the risk of a seizure (this may be sensed by the magnetometer 135) or (ii) that the user appears to be sleeping in a prone position, which may be associated with an increased risk of sudden unexpected death in epilepsy (SUDEP) (this may be sensed by an accelerometer). Such a warning may take any one of several possible forms. For example, the implantable device 100 may send a wireless signal (e.g., a Bluetooth™ signal, or a near field communication (NFC) signal, or a WiFi™ signal) to a nearby external device, such as a mobile telephone or a portable computer (e.g., a laptop or a tablet computer). The external device may then generate an audible alarm (or display a conspicuous warning message on its screen). In other embodiments, the implantable device 100 may include an actuator for alerting the user directly, such as a small motor with an off-center weight secured to its shaft (suitable for producing perceptible vibrations) or an electromagnet (which may also be used to produce perceptible vibrations, or which may be used to tap on the interior of the housing 110 of the implantable device 100).

In some embodiments, a device otherwise like the implantable device 100 may be worn externally (e.g., on the neck or chest of the subject) instead of being implanted. Such a device (i) may include the sensors 105, 135, the controller 120, and the battery 125 and (ii) may, for example, be inductively coupled, through the skin of the subject, to the electrode wires 130 for performing vagus nerve stimulation. The vagus nerve stimulation circuit 115 in such an embodiment may be primarily in the device (with, e.g., only a rectifying circuit being implanted along with the electrode wires 130) or primarily implanted (with power and commands being transmitted by the device to the implanted vagus nerve stimulation circuit 115 through an inductively-coupled connection).

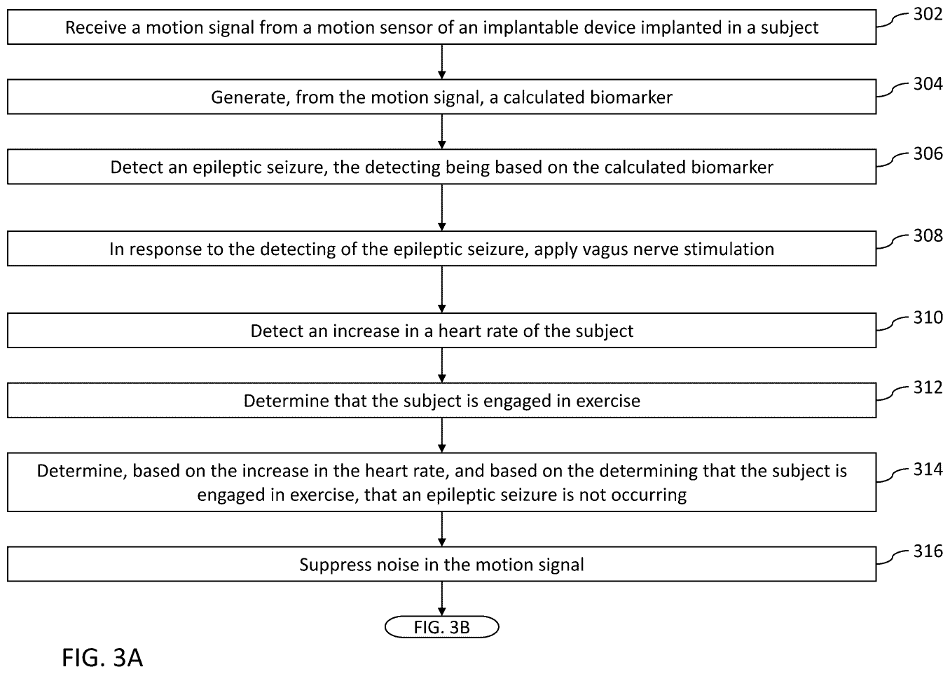

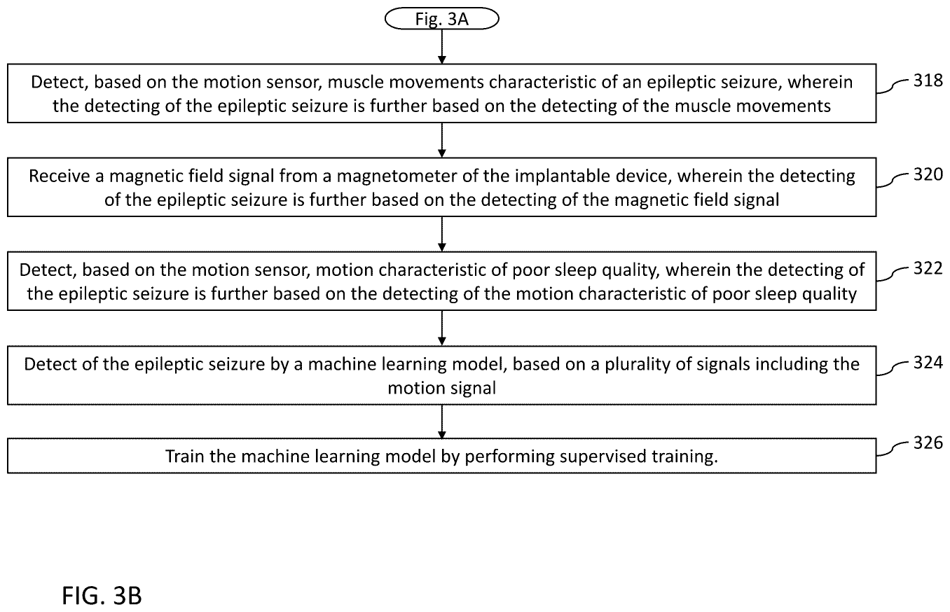

FIGS. 3A and 3B show a method for vagus nerve stimulation, in some embodiments. Although FIGS. 3A and 3B illustrate various operations in the method, embodiments according to the present disclosure are not limited thereto. For example, according to some embodiments, the method may include additional operations or fewer operations, or the order of operations may vary (unless otherwise explicitly stated or implied) without departing from the spirit and scope of embodiments according to the present disclosure.

The method of FIGS. 3A and 3B includes receiving, at 302, a motion signal from a motion sensor of an implantable device implanted in a subject. The motion sensor may, as mentioned above, be or include a simple or compound accelerometer, or a simple or compound gyroscope. As such, the motion signal may be a scalar signal (e.g., representing acceleration along a single axis) or a vector signal (representing, for example, an acceleration vector). The method further includes generating, at 304, from the motion signal, a calculated biomarker. As mentioned above, the calculated biomarker may be, or include, a calculated heart rate, or a calculated respiration rate, or both. The method further includes detecting, at 306, an epileptic seizure, the detecting being based on the calculated biomarker. This detecting may involve, as mentioned above, detecting a sudden increase in the heart rate (e.g., an increase of between 5 and 100 beats per minute over a time interval having a length between 3 seconds and 40 seconds) or detecting a sudden decrease in the respiration rate (e.g., a decrease of between 3 and 20 breaths per minute over a time interval having a length between 3 seconds and 40 seconds). In some embodiments, the calculated biomarker includes both a calculated heart rate and a calculated respiration rate, and both of these signals are calculated based on the signal from a single (simple or composite) motion sensor. As another example, the biomarker may include muscle movements characteristic of an epileptic seizure, and the detecting of the epileptic seizure may include determining that such movements are present at an amplitude that is consistent with the hypothesis that a seizure is occurring. The method further includes, applying, at 308, vagus nerve stimulation (e.g., by the vagus nerve stimulation circuit 115), in response to the detecting of the epileptic seizure. The controller 120 may apply vagus nerve stimulation by instructing the vagus nerve stimulation circuit 115 to apply such stimulation, for example. In some embodiments, the elements of the system except for the electrode wires 130 are all enclosed in a single housing, as illustrated for example, in FIG. 2.

The method may further include, as mentioned above, measures to avoid false detections of seizures, e.g., as a result of heart rate increases due to exercise. For example, the method may include detecting, at 310, an increase in a heart rate of the subject, determining, at 312, (e.g., based on a signal from the motion sensor 105) that the subject is engaged in exercise, and determining, at 314, based on (i) the increase in the heart rate, and based on (ii) the determining that the subject is engaged in exercise, that an epileptic seizure is not occurring. In this example, the rate of increase of the heart rate may be one that, in the absence of an indication that the subject was exercising, would have indicated the occurrence of a seizure.

The motion sensor 105 may be or include, as mentioned above, a micro-electromechanical systems (MEMS) sensor, an accelerometer (e.g., a MEMS accelerometer), or a gyroscope (e.g., a MEMS gyroscope). The method may include suppressing noise in the motion signal, at 316. The method may further include, as mentioned above, detecting, at 318, based on the motion sensor, muscle movements that are characteristic of an epileptic seizure, and the detecting of the epileptic seizure may be further based on the detecting of the muscle movements. The method may further include, as mentioned above, receiving, at 320, a magnetic field signal from a magnetometer of the implantable device 100, wherein the detecting of the epileptic seizure is further based on the detecting of the magnetic field signal. For example, the presence of a strong magnetic field may increase the likelihood that a seizure is occurring, and the system (e.g., the controller 120) may accordingly employ a reduced threshold in determining, for example, whether a measured rate of increase of the heart rate of the subject is an indication that a seizure is occurring.

The method may further include, as mentioned above, detecting, at 322, based on the motion sensor, motion that is characteristic of poor sleep quality, wherein the detecting of the epileptic seizure is further based on the detecting of the motion that is characteristic of poor sleep quality. For example, if the controller 120 detects, while the subject appears to be sleeping, frequent changes in position, the controller 120 may determine that the subject is experiencing low-quality sleep; this determination may subsequently be taken into account when determining whether a seizure is occurring (because poor sleep quality may increase the likelihood of a seizure). In some embodiments, as mentioned above, the detecting of an epileptic seizure comprises detecting, at 324, of the epileptic seizure by (an inference operation of) a machine learning model, based on a plurality of signals including the motion signal; the plurality of signals may further include a magnetic field signal. The method may further include, as mentioned above, training, at 326, the machine learning model (before performing inference operations) by performing supervised training with training data comprising a plurality of labeled data elements, each labeled data element being labeled with an indicator of whether a seizure was occurring when the data element was collected.

The systems and methods disclosed herein may result in more reliable automatic detection of epileptic seizures, or more effective mitigation of such seizures, than other available systems and methods; as such, the systems and methods disclosed herein contain (e.g., are) improvements to the technologies of epileptic seizure detection and epileptic seizure mitigation.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X−Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software (including at least some hardware), employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Further, the use of “may” when describing embodiments of the inventive concept refers to “one or more embodiments of the present disclosure”. Also, the term “exemplary” is intended to refer to an example or illustration. As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for vagus nerve stimulation have been specifically described and illustrated herein, many modifications and variations will be apparent to those skilled in the art. Accordingly, it is to be understood that a system and method for vagus nerve stimulation constructed according to principles of this disclosure may be embodied other than as specifically described herein. The invention is also defined in the following claims, and equivalents thereof.

1. A method, comprising:
receiving a motion signal from a motion sensor of an implantable device implanted in a subject;
generating, from the motion signal, a calculated biomarker;
detecting an epileptic seizure, the detecting being based on the calculated biomarker; and
in response to the detecting of the epileptic seizure, applying, by the implantable device, vagus nerve stimulation.

2. The method of claim 1, wherein:
the calculated biomarker comprises a calculated heart rate or a calculated respiration rate; and
the detecting, based on the calculated biomarker, of the epileptic seizure, comprises detecting the epileptic seizure based on an increase or decrease in the calculated heart rate or based on an increase or decrease in the calculated respiration rate.

3. The method of claim 2, wherein:
the calculated biomarker comprises a calculated respiration rate; and
the detecting, based on the calculated biomarker, of the epileptic seizure, comprises detecting the epileptic seizure based on an increase or decrease in the calculated respiration rate.

4. The method of claim 3, wherein:
the detecting of the epileptic seizure comprises detecting a decrease in the calculated respiration rate, and
the detecting of the decrease in the calculated respiration rate comprises performing frequency tracking of the calculated respiration rate with an infinite impulse response adaptive notch filter tuned to the calculated respiration rate.

5. The method of claim 1, wherein:
the generating of the calculated biomarker comprises calculating a heart rate; and
the detecting, based on the calculated biomarker, of the epileptic seizure, comprises detecting the epileptic seizure based on an increase in the calculated heart rate.

6. The method of claim 5, wherein the calculating of the heart rate comprises performing a method selected from the group consisting of linear filtering, numerical differentiation, application of a memoryless nonlinear transform, low pass filtering, peak detection, and combinations thereof.

7. The method of claim 1, further comprising:
detecting an increase in a heart rate of the subject;
determining that the subject is engaged in exercise; and
determining, based on the increase in the heart rate, and based on the determining that the subject is engaged in exercise, that an epileptic seizure is not occurring.

8. The method of claim 1, further comprising detecting, based on the motion sensor, muscle movements characteristic of an epileptic seizure, wherein the detecting of the epileptic seizure is further based on the detecting of the muscle movements.

9. The method of claim 8, wherein the detecting of the muscle movements comprises detecting a period of high amplitude signals in a frequency band characteristic of shaking encountered during clonic seizures.

10. The method of claim 1, further comprising receiving a magnetic field signal from a magnetometer of the implantable device, wherein the detecting of the epileptic seizure is further based on the detecting of the magnetic field signal.

11. The method of claim 1, further comprising detecting, based on the motion sensor, motion characteristic of poor sleep quality, wherein the detecting of the epileptic seizure is further based on the detecting of the motion characteristic of poor sleep quality.

12. The method of claim 11, wherein the detecting of the motion characteristic of poor sleep quality comprises detecting motion corresponding to a position change of the subject while the subject is lying down.

13. The method of claim 1, wherein:
the detecting of the epileptic seizure comprises detecting the calculated biomarker passing a threshold, and
the threshold is based on a history of the calculated biomarker.

14. The method of claim 1, wherein the detecting of the epileptic seizure comprises detecting of the epileptic seizure by a machine learning model, based on a plurality of signals including the motion signal.

15. The method of claim 14, wherein the plurality of signals further includes a magnetic field signal.

16. The method of claim 14, further comprising training the machine learning model by performing supervised training with training data comprising a plurality of labeled data elements, each labeled data element being labeled with an indicator of whether a seizure was occurring when the data element was collected.

17. The method of claim 1, wherein the implantable device comprises a housing having a biocompatible outer surface and containing the motion sensor and a vagus nerve stimulation circuit.

18. The method of claim 1, wherein the motion sensor is a micro-electromechanical systems (MEMS) sensor.

19. The method of claim 1, wherein the motion sensor comprises an accelerometer.

20. The method of claim 1, wherein the motion sensor comprises a gyroscope.

21.-40. (canceled)
